(19) United States
(12) Patent Application Publication
(10) Pub. No.: US 2020/0315541 A1
(43) Pub. Date: Oct. 8, 2020

(54) Gastrointestinal Capsule and Treatment Method

(71) Applicant: VIBRANT LTD., Yokneam (IL)

(72) Inventors: Lior BEN-TSUR, Netanya (IL); Camille MORLIERE, Hadera (IL); Ronny SHABBAT, Kibbutz Yizrael (IL)

(21) Appl. No.: 16/823,035

(22) Filed: Mar. 18, 2020

Related U.S. Application Data
(63) Continuation-in-part of application No. 15/619,493, filed on Jun. 11, 2017.
Application No. 15/619,493 is a continuation of application No. 15/882,536, filed on Jan. 29, 2018.
Application No. 15/619,493 is a continuation of application No. 14/461,414, filed on Aug. 17, 2014, now Pat. No. 9,707,150.
Application No. 14/461,414 is a continuation-in-part of application No. PCT/IB2013/000203, filed on Feb. 17, 2012.
(60) Provisional application No. 62/451,831, filed on Jan. 30, 2017.
(60) Provisional application No. 61/930,972, filed on Jan. 24, 2014.

(30) Foreign Application Priority Data
Feb. 16, 2012 (GB) 1202706.6

(51) Int. Cl. A61B 5/00; A61B 5/07

(52) U.S. Cl. CPC A61B 5/6861; A61H 2201/1207; A61B 5/073

(57) ABSTRACT

A gastrointestinal capsule and treatment methods utilizing such a capsule, the capsule including: (a) a capsule housing having a longitudinal axis; (b) at least one thrusting mechanism, disposed within the housing, the thrusting mechanism adapted to exert forces on the housing such that, when the capsule is disposed within a gastrointestinal tract of a user, and the mechanism is in an active mode, the gastrointestinal capsule exerts forces against, or in a direction of, the walls of the tract; and (c) a power supply adapted to power the mechanism. The gastrointestinal capsule is used in a method for treating a sensation of straining during defecating and/or for increasing a Bristol stool score of fecal matter of a subject by the gastrointestinal capsule being ingested by the subject and activated in a targeted zone of the gastrointestinal tract of the subject.

### REFERENCE TO RELATED APPLICATIONS

This application is a continuation in part of U.S. patent application Ser. No. 15/619,493, filed Jun. 11, 2017, which is a continuation of U.S. patent application Ser. No. 14/461,414 filed Aug. 17, 2014, now U.S. Pat. No. 9,707,150. U.S. Pat. No. 9,707,150 is a continuation in part of PCT/IB2013/000203, filed Feb. 17, 2013, which draws priority from UK Patent Application No. GB1202706.6, filed Feb. 16, 2012 and from U.S. Provisional Patent Application Ser. No. 61/602,093, filed Feb. 23, 2012.

This application is also a continuation in part of U.S. patent application Ser. No. 15/882,536, filed Jan. 29, 2018, which gains priority from U.S. Provisional Patent Application No. 62/451,831 filed Jan. 30, 2017.

U.S. patent application Ser. Nos. 15/619,493; 14/461,414; and Ser. No. 15/882,536, U.S. Pat. No. 9,707,150; PCT Patent Application No. PCT/IB2013/000203; UK Patent Application GB1202706.6; and U.S. Provisional Patent Applications 61/602,093 and 62/451,831 are all incorporated by reference for all purposes as if fully set forth herein.

### FIELD AND BACKGROUND OF THE INVENTION

The present invention relates to gastrointestinal capsules (GICs).

The present invention relates in general to methods for reducing a straining sensation and increasing Bristol stool scores, particularly to methods for reducing a straining sensation of a human subject during defecating and increasing Bristol stool scores of fecal matter of the subject using a vibrating ingestible capsule.

Intestinal constipation is a widespread gastrointestinal motility disorder. Various treatment programs are known, employing dietary modifications and supplements, laxatives, and suppositories. In severe cases, surgery may be indicated. Constipation may be considered a symptom, and care must be taken, in treating the symptom, not to exacerbate or aggravate the general condition of the patient. Thus, by way of example, the frequent or long-term use of laxatives may be detrimental, as such laxatives may compromise the ability of the body to independently effect bowel movements.

An ingestible gastrointestinal capsule for mechanically stimulating a segment of the gastrointestinal wall is disclosed by U.S. Patent Publication No. 20090318841, which is incorporated by reference for all purposes as if fully set forth herein.

However, the present inventor has recognized a need for improved gastrointestinal capsules and treatment methods utilizing such capsules.

### SUMMARY OF THE INVENTION

According to the teachings of the present invention there is provided a gastrointestinal capsule (GIC) including: (a) a capsule housing having a longitudinal axis; (b) at least one thrusting mechanism, disposed within the housing, the thrusting mechanism adapted to exert radial forces on the housing, in a radial direction with respect to the axis, such that, when the capsule is disposed within a gastrointestinal tract of a user, and the mechanism is in an active mode, the gastrointestinal capsule exerts forces against, or in a direction of the walls of the tract; and (c) a power supply adapted to power the mechanism, wherein a ratio of the radial forces to axial forces exerted in an axial direction with respect to the axis, on the housing, by the thrusting mechanism, is at least 1:1, at least 1.25:1, at least 1.5:1, at least 2:1, at least 3:1, at least 4:1, or at least 5:1.

According to yet another aspect of the present invention there is provided a gastrointestinal capsule including: (a) a housing; (b) a thrusting mechanism, disposed within the housing, the mechanism having an active mode and a passive mode, with respect to the active mode, the mechanism adapted to exert a radial force on the housing, whereby, when the capsule is disposed within a gastrointestinal tract of a user, and the mechanism is in the active mode, the gastrointestinal capsule stimulates the walls of the tract; and (c) a battery adapted to power the mechanism, wherein the active mode includes a series of at least two pulses of the radial force, the series having a first duration, the passive mode has a second duration, and wherein an activation cycle is defined by the series of pulses followed by the second duration, and wherein the first duration is within a range of 1-10 seconds.

According to still further features in the described preferred embodiments, the ratio is at most 20:1, at most 12:1, at most 10:1, at most 8:1, at most 7:1, or at most 6:1.

According to still further features in the described preferred embodiments, the ratio is within a range of 1:1 to 15:1, 2.5:1 to 15:1, 2.5:1 to 10:1, 2.5:1 to 8:1, or 2.5:1 to 6:1.

According to still further features in the described preferred embodiments, the at least one thrusting mechanism includes an axial perturbation arrangement having: (i) a motor electrically connected to the power supply; and (ii) an urging mechanism, associated with, and driven by, the motor, the urging mechanism adapted to exert the axial forces.

According to still further features in the described preferred embodiments, the urging mechanism includes: a motor shaft, disposed at least partially in a direction along the longitudinal axis, the shaft being operatively connected to, and driven by, the motor; and a thrusting weight associated with the shaft, the urging mechanism further adapted to at least periodically urge the weight along the shaft, to deliver the axial forces.

According to still further features in the described preferred embodiments, the urging mechanism further includes a stopper or cap, adapted to receive a distal end of the shaft, the stopper or cap impinging against an inner wall of the capsule housing.

According to still further features in the described preferred embodiments, the urging mechanism further includes a spring associated with the motor shaft, the urging mechanism being adapted such that, in a first state, the spring is compressed, and such that, in a second state, the spring is released against the weight, to urge the weight along the shaft, to deliver the axial forces against the capsule housing.

According to still further features in the described preferred embodiments, the motor shaft passes through the weight, the motor shaft has an external interrupted thread, and the weight has a threaded internal surface generally complementary to a threading of the interrupted thread, whereby, in the first state, the external interrupted thread engages the threaded internal surface, and in the second state, the threaded internal surface is disengaged and longitudinally free with respect to the interrupted thread.

According to still further features in the described preferred embodiments, the motor shaft passes through the weight, the weight being adapted to turn with the shaft, the motor shaft having an external interrupted thread, and the weight having a threaded internal surface generally complementary to a threading of the interrupted thread, the urging mechanism being further adapted such that in the first state, the external interrupted thread engages the threaded internal surface to compress the spring, and in a second state, the threaded internal surface is disengaged and longitudinally free with respect to the interrupted thread, such that the spring is released against the weight.

According to still further features in the described preferred embodiments, the thrusting mechanism includes a rotatably mounted eccenter, the thrusting mechanism being adapted to rotate the eccenter to exert the radial forces.

According to still further features in the described preferred embodiments, the thrusting mechanism is configured to have the active mode and a passive mode with respect to the active mode, the active mode including a series of at least two pulses of the radial forces, wherein the series has a first duration, the passive mode has a second duration, and wherein the second duration exceeds the first duration.

According to still further features in the described preferred embodiments, the first duration and the second duration define an activation cycle, the thrusting mechanism being configured such that the activation cycle has a period within a range of 5-60 seconds, 7-40 seconds, 8-30 seconds, 10-30 seconds, or 12-25 seconds.

According to still further features in the described preferred embodiments, the first duration and the second duration define an activation cycle, the thrusting mechanism being configured such that the activation cycle has a period of at least 5, at least 6, at least 7, at least 8, at least 10, at least 12, or at least 15 seconds, and/or at most 60, at most 40, at most 30, at most 25, or at most 20 seconds.

According to still further features in the described preferred embodiments, the thrusting mechanism is configured such that the first duration is within a range of 1-10 seconds, 2-8 seconds, or 2.5-6 seconds.

According to still further features in the described preferred embodiments, the thrusting mechanism is configured such that a net force exerted by the capsule on an external environment is at least 400 grams force, at least 450 grams force, at least 500 grams force, or at least 600 grams force.

According to still further features in the described preferred embodiments, the thrusting mechanism is configured such that the net force is an instantaneous net force of at least 800 grams force, at least 1000 grams force, at least 1200 grams force, at least 1400 grams force, or at least 1500 grams force.

According to still further features in the described preferred embodiments, the thrusting mechanism is configured to exert the radial forces on the housing to attain a vibrational frequency within a range of 12 Hz to 80 Hz.

According to still further features in the described preferred embodiments, the thrusting mechanism is configured such that the range is 12 Hz to 70 Hz, 15 Hz to 60 Hz, 15 Hz to 50 Hz, 18 Hz to 45 Hz, or 18 Hz to 40 Hz.

According to still further features in the described preferred embodiments, the thrusting mechanism is configured such that the vibrational frequency is at least 15 Hz, at least 18 Hz, at least 20 Hz, or at least 22 Hz.

According to still further features in the described preferred embodiments, the thrusting mechanism is configured such that the vibrational frequency is at most 75 Hz, at most 70 Hz, at most 60 Hz, at most 50 Hz, at most 45 Hz, or at most 40 Hz.

According to still further features in the described preferred embodiments, the axial arrangement is adapted to exert the axial forces in opposite directions.

According to still further features in the described preferred embodiments, the axial arrangement is adapted to deliver at least a portion of the axial forces in a knocking mode.

According to still further features in the described preferred embodiments, the thrusting mechanism has a first individual motor for delivering the radial forces and a second individual motor for delivering the axial forces.

According to still further features in the described preferred embodiments, the first individual motor and the second individual motor are disposed on different sides of the capsule, with respect to the axis.

According to still further features in the described preferred embodiments, the thrusting mechanism is adapted such that when the capsule is disposed within the tract, and the mechanism is in the active mode, the capsule stimulates the walls of the tract.

According to still further features in the described preferred embodiments, the thrusting mechanism includes a controller, electrically attached to the power supply, the controller adapted to control the thrusting mechanism.

According to still further features in the described preferred embodiments, the controller is physically isolated from all motors within the housing.

According to still further features in the described preferred embodiments, the controller is physically isolated, by at least 2 mm, from all motors within the housing.

According to still further features in the described preferred embodiments, the thrusting mechanism is adapted to exert a radial force on the housing, whereby, when the capsule is disposed within a gastrointestinal tract of a user, and the thrusting mechanism is in the active mode, the gastrointestinal capsule induces a peristaltic wave in the walls of the tract.

According to still further features in the described preferred embodiments, the length of the GIC is at most 28 mm, at most 26 mm, at most 25 mm, at most 24 mm, at most 22 mm, at most 20 mm, at most 18 mm, at most 15 mm, or at most 12 mm.

According to still further features in the described preferred embodiments, the weight of the GIC is at most 25 grams, at most 22 grams, at most 20 grams, at most 17 grams, at most 15 grams, at most 12 grams, or at most 10 grams.

According to yet another aspect of the present invention there is provided a therapeutic method for mechanically stimulating a wall of a segment of a mammalian gastrointestinal tract of a user by means of a gastrointestinal capsule, the method including: (a) providing the gastrointestinal capsule; (b) administering at least one treatment session, each treatment session including: (i) delivering the gastrointestinal capsule into the tract; and (ii) effecting activation of a thrusting mechanism of the gastrointestinal capsule to achieve mechanical stimulation of the wall of the gastrointestinal tract.

According to still further features in the described preferred embodiments, the at least one treatment session includes a plurality of the treatment sessions.

According to still further features in the described preferred embodiments, at least one of the treatment sessions is administered per week, over a treatment period extending for at least two weeks, at least three weeks, at least four weeks, at least five weeks, at least six weeks, or at least eight weeks.

According to still further features in the described preferred embodiments, at least 1.5, at least 1.75, at least 2, at least 2.5, or at least 3 of the treatment sessions is administered per week of the treatment period.

According to still further features in the described preferred embodiments, a frequency of the treatment sessions administered to the user is within a range of 1.5 to 6 per week of the treatment period.

According to still further features in the described preferred embodiments, the frequency is within a range of 1.5 to 5, 1.5 to 4, 1.5 to 3.5, 1.5 to 3, 2 to 6, 2 to 5, 2 to 4, 2 to 3.5, or 2 to 3, per week of the treatment period.

According to still further features in the described preferred embodiments, within each the treatment session, the activation of the thrusting mechanism is performed for a duration effective to achieve the mechanical stimulation of the wall of the gastrointestinal tract.

According to still further features in the described preferred embodiments, within each treatment session, the activation of the thrusting mechanism is performed for a duration effective to increase a frequency of spontaneous bowel movements of the user.

According to still further features in the described preferred embodiments, within each treatment session, the activation of the thrusting mechanism is performed for a duration effective to increase a frequency of spontaneous bowel movements of the user by at least 25%, at least 50%, at least 75%, or at least 100%.

According to still further features in the described preferred embodiments, within each treatment session, the activation of the thrusting mechanism is performed for a duration effective to at least partially relieve a condition of constipation of the user.

According to still further features in the described preferred embodiments, within each treatment session, the activation of the thrusting mechanism is performed for a duration effective to completely relieve a condition of constipation of the user.

According to still further features in the described preferred embodiments, the vibration frequency and relaxation period may be varied, within a single treatment period, in order to prevent habituation.

According to still further features in the described preferred embodiments, the delivering of the GIC is performed via oral insertion.

According to still further features in the described preferred embodiments, the delivering of the GIC is performed by inserting the GIC into the tract via a rectal opening of the user.

In accordance with an embodiment of the present invention, there is provided a method of treating a sensation of straining while defecating in a human subject using a gastrointestinal capsule adapted to transit an alimentary canal of the subject, the capsule having:

a housing;

a battery, disposed within the housing; and

a vibrating agitation mechanism, powered by the battery, the vibrating agitation mechanism adapted such that, in a first vibrating mode of operation, the housing exerts vibrations on an environment surrounding the capsule, the method including:

(a) ingesting the gastrointestinal capsule; and
(b) controlling the vibrating agitation mechanism such that at least a portion of the first vibrating mode of operation occurs when the capsule is disposed within a targeted zone within a gastrointestinal tract of the subject, so as to alleviate or dissipate the sensation of straining while defecating.

In accordance with another embodiment of the present invention, there is provided a method of increasing a Bristol stool score of fecal matter defecated by a subject using a gastrointestinal capsule adapted to transit an alimentary canal of the subject, the capsule having:

a housing;

a battery, disposed within the housing; and

a vibrating agitation mechanism, powered by the battery, the vibrating agitation mechanism adapted such that, in a first vibrating mode of operation, the housing exerts vibrations on an environment surrounding the capsule, the method including:

(a) ingesting the gastrointestinal capsule; and
(b) controlling the vibrating agitation mechanism such that the first vibrating mode of operation occurs when the capsule is disposed within a targeted zone within a gastrointestinal tract of the subject, so as to increase the Bristol stool score of fecal matter defecated by the subject.

In some embodiments, the targeted zone includes an intestinal section of the gastrointestinal tract of the subject. In some embodiments, the targeted zone includes a section of a large intestine of the subject. In some embodiments, the targeted zone includes a rectal section of the gastrointestinal tract of the subject.

In some embodiments, controlling includes pre-setting an activation time delay of the capsule, prior to ingesting. In some embodiments, the activation time delay is in the range of 2 hours to 48 hours, 2 hours to 42 hours, 2 hours to 36 hours, 2 hours to 30 hours, 2 hours to 24 hours, 3 hours to 24 hours, 4 hours to 24 hours, 4 hours to 20 hours, 4 hours to 18 hours, 4 hours to 16 hours, 4 hours to 14 hours, 4 hours to 12 hours, 6 hours to 12 hours, or 6 hours to 10 hours.

In some embodiments, the subject is a particular subject and pre-setting of the activation time delay is according to a measured or estimated transit time of chyme along the gastrointestinal tract of the particular subject.

In some embodiments the method further includes, prior to pre-setting of the activation time delay, obtaining information relating to the measured or estimated transit time of chime along the gastrointestinal tract of the particular subject.

In some embodiments, the vibrating agitation mechanism includes at least a radial agitation mechanism adapted, in the first vibrating mode of operation, to exert radial forces on the housing, in a radial direction with respect to a longitudinal axis of the housing, thereby to cause the vibrations of the housing. In some embodiments, the radial agitation mechanism includes unbalanced weight attached to a shaft of an electric motor powered by the battery.

In some embodiments, the vibrating agitation mechanism includes at least an axial agitation mechanism adapted, in the first vibrating mode of operation, to exert axial forces on the housing, in an axial direction with respect to a longitudinal axis of the housing, thereby to cause the vibrations of the housing. In some embodiments, the axial agitation mechanism includes an electric motor powered by the battery and an urging mechanism, associated with, and driven by, the electric motor, the urging mechanism adapted to exert the axial forces. In some embodiments, the urging mechanism is adapted to exert the axial forces in opposite directions. In some embodiments, the urging mechanism is adapted to deliver at least a portion of the axial forces in a knocking mode.

In some embodiments, the vibrating agitation mechanism is adapted in the first vibrating mode of operation, to exert radial forces on the housing in a radial direction with respect to a longitudinal axis of the housing and to exert axial forces on the housing in an axial direction with respect to the longitudinal axis of the housing, thereby to cause the vibrations of the housing. In some embodiments, the vibrating agitation mechanism includes a radial agitation mechanism adapted to exert the radial forces and a separate axial agitation mechanism adapted to exert the axial forces. In some other embodiments, the vibrating agitation mechanism includes a single agitation mechanism adapted to exert the radial forces and the axial forces.

In some embodiments, the vibrating mode of operation including a plurality of cycles, each of the cycles including a vibration duration followed by a repose duration, wherein the housing exerts the vibrations during the vibration duration.

In some embodiments, the number of vibration cycles per hour is in the range of 20 to 400, 40 to 400, 60 to 400, 80 to 400, 40 to 380, 60 to 380, 80 to 380, 40 to 360, 60 to 360, 80 to 360, 100 to 360, 100 to 330, 100 to 300, 100 to 280, 100 to 250, 100 to 220, 100 to 200, 120 to 300, 120 to 280, 120 to 250, 120 to 220, 120 to 200, 150 to 300, 150 to 280, 150 to 250, 150 to 220, 150 to 200, 170 to 300, 170 to 250, 170 to 220, or 170 to 200.

In some embodiments, the repose duration is greater than the vibration duration.

In some embodiments, the vibration duration is in the range of 0.1 second to 10 seconds, 1 second to 10 seconds, 1 second to 9 seconds, 2 seconds to 9 seconds, 3 seconds to 9 seconds, 3 seconds to 8 seconds, 3 seconds to 7 seconds, 3 seconds to 6 seconds, or 4 seconds to 6 seconds.

In some embodiments, the repose duration is in the range of 1 second to 180 seconds, 3 seconds to 180 seconds, 5 seconds to 180 seconds, 5 seconds to 150 seconds, 5 seconds to 120 seconds, 8 seconds to 100 seconds, 8 seconds to 30 seconds, 10 seconds to 80 seconds, 10 seconds to 70 seconds, 10 seconds to 60 seconds, 10 seconds to 50 seconds, 10 seconds to 40 seconds, 10 seconds to 30 seconds, 10 seconds to 20 seconds, or 15 seconds to 20 seconds.

In some embodiments, a duration of each of the plurality of cycles is in the range of 1.1 seconds to 200 seconds, 5 seconds to 200 seconds, 10 seconds to 200 seconds, 10 seconds to 150 seconds, 10 seconds to 100 seconds, 10 seconds to 80 seconds, 10 seconds to 50 seconds, 10 seconds to 40 seconds, 10 seconds to 30 seconds, 15 seconds to 50 seconds, 15 seconds to 40 seconds, 15 seconds to 30 seconds, or 15 seconds to 25 seconds.

In some embodiments, a cumulative duration of the vibrating mode of operation is in the range of 1 hour to 12 hours, 2 hours to 10 hours, 2 hours to 8 hours, 2 hours to 6 hours, 2 hours to 4 hours, or 2 hours to 3 hours. In some embodiments, the cumulative duration is dependent on properties of the battery.

In some embodiments, the vibrating agitation mechanism is configured such that a net force exerted by the housing on the environment is in the range of 50 grams-force to 600 grams-force.

In some embodiments, the vibrating agitation mechanism is configured to exert the forces on the housing to attain a vibrational frequency within a range of 10 Hz to 650 Hz, 15 Hz to 600 Hz, 20 Hz to 550 Hz, 30 Hz to 550 Hz, 50 Hz to 500 Hz, 70 Hz to 500 Hz, 100 Hz to 500 Hz, 130 Hz to 500 Hz, or 150 Hz to 500 Hz.

In some embodiments, controlling of the vibrating agitation mechanism is effected so as to effect a mechanical stimulation of the wall of the gastrointestinal tract in the targeted zone.

In some embodiments, the subject is a subject who has experienced at most one of the following symptoms over the preceding 3 months:

fewer than three bowel movements per week;

straining;

lumpy or hard stools;

sensation of anorectal obstruction;

sensation of incomplete defecation; and

manual maneuvering required to defecate.

In some embodiments, the subject is a subject who has experienced at most one of the following symptoms over the preceding 3 months:

fewer than three bowel movements per week;

straining during more than 25% of defecations;

lumpy or hard stools in more than 25% of defecations;

sensation of incomplete defecation in more than 25% of defecations;

sensation of anorectal obstruction in more than 25% of defecations; and

manual maneuvering required to facilitate more 25% of defecations.

In some embodiments, the subject is a constipation free subject.

In some embodiments, the subject suffers from emotional stress, or has psychosomatically induced symptoms.

In some embodiments, the ingesting and controlling together form a treatment session, and wherein the method includes administering to the subject at least one the treatment session.

In some embodiments, administering to the subject at least one treatment session includes administering to the subject a plurality of treatment sessions.

In some embodiments, administering a plurality of treatment sessions includes administering at least one the treatment session per week, over a treatment period of at least two weeks, at least at least three weeks, at least four weeks, at least five weeks, at least six weeks, or at least eight weeks.

In some embodiments, administering at least one treatment session per week includes administering 1 to 7 treatment sessions per week, 3 to 14 treatment sessions per two weeks, 2 to 7 treatment sessions per week, 5 to 14 treatment sessions per two weeks, 3 to 7 treatment sessions per week, 7 to 14 treatment sessions per two weeks, 4 to 7 treatment sessions per week, or 5 to 7 treatment sessions per week.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. Throughout the drawings, like-referenced characters are used to designate like elements.

In the drawings:

FIG. 1 is a schematic exploded view of a GIC according to some embodiments of the present invention;

FIG. 2A is a cut-open, perspective view of the GIC provided in FIG. 1;

FIG. 2B is a side view of the cut-open GIC of FIG. 2A;

FIG. 3A is a schematic, top view of a cut-open GIC according to some embodiments of the present invention;

FIG. 3B is a side view of the cut-open GIC of FIG. 3A;

FIG. 3C is a partial perspective view of the cut-open GIC of FIG. 3A;

FIG. 3D is another partial view of cut-open GIC 300, showing a magnified perspective view of an external interrupted thread of screw shaft, according to one embodiment of the invention;

FIG. 4 is a schematic block diagram of a vibrating ingestible capsule for reducing a straining sensation of a human subject during defecating and improving Bristol stool scores of fecal matter of the subject according to an embodiment of the present invention;

FIG. 5 is a schematic flowchart of a method of treatment for reducing a straining sensation of a human subject during defecating and improving Bristol stool scores of fecal matter of the subject according to the present invention, the treatment being based one use of an ingestible vibrating gastrointestinal capsule;

FIG. 6 is a graphic representation of reduction in a straining sensation in subjects treated by the method of FIG. 5;

FIG. 7 is a graphic representation of a Bristol stool score of fecal matter of subjects treated by the method of FIG. 5;

FIG. 8 is a graphic representation of reduction in a straining sensation in subjects treated by the method of FIG. 5 as compared to subjects treated with a sham capsule; and

FIG. 9 is a graphic representation of a Bristol stool score of fecal matter of subjects treated by the method of FIG. 5 as compared to subjects treated with a sham capsule.

### DESCRIPTION OF THE PREFERRED EMBODIMENTS

The principles and operation of the inventive gastrointestinal capsules, and the treatment methods utilizing such capsules, may be better understood with reference to the drawings and the accompanying description.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

For the purposes of this application, the term “subject” relates to a human.

For the purposes of this application, the term “vibrating ingestible capsule” relates to an ingestible capsule adapted to at least intermittently vibrate, for a cumulative duration of at least one minute, in accordance with a vibration protocol of the capsule.

For the purposes of this application, the term “intermittently activated vibrating agitation mechanism” refers to a vibration engine that vibrates and is operative at certain times, and does not vibrate at other times, the activation times being selected by a processor or other control unit controlling the vibration engine.

For the purposes of this application, the term “vibration protocol” relates to a protocol specifying vibration parameters of an intermittently activated vibrating agitation mechanism of a vibrating ingestible capsule. Typically, the vibration protocol relates to an activation delay for initiating vibration (a duration between activation of the capsule and the first activation of the vibration engine), a vibration rate (number of vibration cycles per hour), a vibration duration and a repose duration for each vibration cycle, a vibration frequency, an amount of force exerted by the vibrations, and the like.

For the purposes of this application, the term “treatment procedure” relates to parameters of a treatment utilizing vibrating ingestible capsules, which are typically defined by a treating physician or medical practitioner. For example, the treatment procedure may include the number of capsule to be taken within a specific time duration (e.g. 3 capsules per week, 2 capsules per day), the frequency at which capsules should be taken, the time of day at which capsules should be taken, whether the capsule should be taken with or without food, and the like.

For the purpose of this application, the term “treatment protocol” relates to all aspects of treatment of a subject with a vibrating ingestible capsule, and includes the treatment procedure as well as the vibration protocol to be used for treating the subject.

For the purpose of the application, the term “transit time” relates to the amount of time it takes for a quantum of food or chyme to move a predetermined distance along the gastrointestinal tract of a particular subject. For example, the transit time may be the amount of time it takes a quantum of chyme to move from the duodenum to the rectum of the subject. The term transit time may relate to a transit time along the whole gastrointestinal tract, from the subject ingesting a food till chyme of that food reaches the rectum, or may relate to transit time within a segment of the gastrointestinal tract, such as the time it takes food from swallowing thereof till it passes from the stomach into the duodenum.

For the purpose of this application, the terms “alleviate sensation of straining” and “dissipate sensation of straining” relate to providing a treatment, such that by the end of a treatment period, may be at least 2 weeks, at least 3 weeks, at least 4 weeks, at least 6 weeks, or at least 8 weeks, the sensation of straining sensed by the subject is at least one point lower than at the beginning of the treatment period, and/or a sensation of straining while defecating occurs 5% to 15% fewer times than at the beginning of the treatment period.

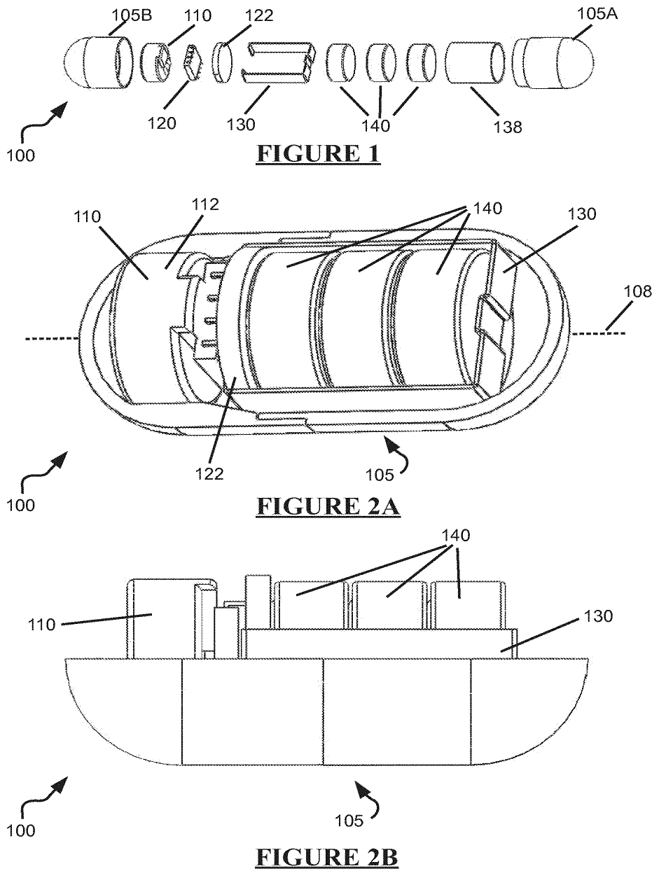

For the purpose of this application, the terms “increase Bristol stool score” relate to providing a treatment, such that by the end of a treatment period, may be at least 2 weeks, at least 3 weeks, at least 4 weeks, at least 6 weeks, or at least 8 weeks, a Bristol stool score of fecal matter, as sensed by the subject, is at least 1 point higher than at the beginning of the treatment period, in at least 5%, at least 10%, at least 15%, at least 20%, or at least 25% of the bowel movements. Referring now to the drawings, FIG. 1 is a schematic block diagram of a vibrating ingestible capsule for reducing a straining sensation of a human subject during defecating and improving Bristol stool scores of fecal matter of the subject according to embodiments of the present invention.

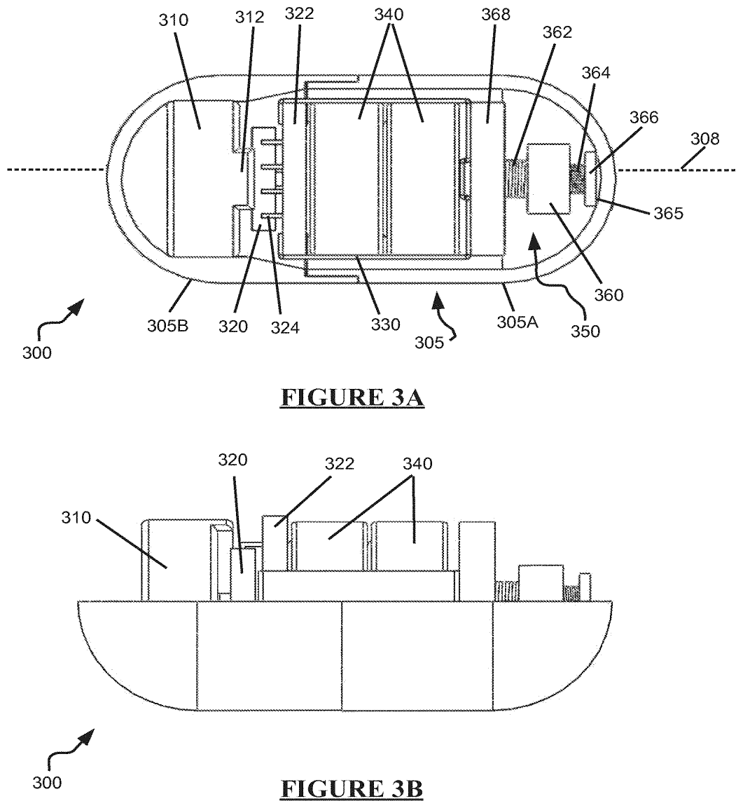

FIG. 1 is a schematic exploded view of a GIC 100 according to some embodiments of the present invention. GIC 100 may include a capsule housing or shell 105 (best seen in FIG. 2A) having complementary (e.g., male and female) components 105A, 105B. Within the capsule housing may be disposed a thrusting mechanism that may include a motor 110 and a circuit board 122 having a CPU, microprocessor or controller 120. Within the capsule housing may further be disposed a power supply such as at least one battery 140, an electrically conductive bridge such as metal bridge 130, and an insulator 138.

A cut-open, perspective view of GIC 100 is provided in FIG. 2A. Three disc-shaped batteries 140 and circuit board 122 may be held together by metal bridge 130. Bridge 130 may be adapted to make electrical contact with a broad face of the battery distal to circuit board 122, and may provide power to circuit board 122. Batteries 140 may also power motor 110, e.g., via conducting wires (not shown) attached to circuit board 122.

The thrusting mechanism may be adapted to deliver to exert radial forces on capsule housing 105. In one embodiment, motor 110 is an eccentric motor having an eccentric weight 112. As motor 110 spins in a generally normal fashion with respect to a longitudinal axis 108 of GIC 100, radial forces are exerted on housing 105.

A side view of the cut-open GIC 100 is provided in FIG. 2B.

The inventive GIC is adapted such that, after ingestion thereof, the GIC is carried by bodily forces through the upper and lower gastrointestinal tracts. Ultimately, the GIC may be naturally evacuated along with the stool.

In accordance with some embodiments of the present invention is provided the GIC may be adapted to repeatedly vibrate within the gastrointestinal walls of the user. The GIC may be automatically activated at a predefined time following ingestion. Similarly, a timing mechanism of (or associated with) CPU 120 may be initiated at, or prior to, ingestion.

In accordance with some embodiments of the present invention, activation of the GIC may be set to automatically occur 2 to 12 hours, 2 to 10 hours, or 2 to 8 hours following ingestion, and more typically, 6 to 10 hours or 6 to 8 hours following ingestion. Such a (typically pre-determined) time delay may match the transit time in which the GIC reaches the large bowel via the upper gastrointestinal tract. The transit time within the large bowel may be significantly longer, in the range of 2 to 5 days, depending on whether the transit time is normal or prolonged, as in cases of constipation. In such cases, the time delay for activation may range between 6 and 24 hours.

Once activated, the inventive GICs may be adapted to agitate for at least 15 minutes, at least 30 minutes, at least 1 hour, at least 1.5 hours, at least 2 hours, at least 2.5 hours, or at least 3 hours, including intermittent periods of rest. Typically, the inventive GICs may be adapted to agitate for less than 8 hours, including intermittent periods of rest.

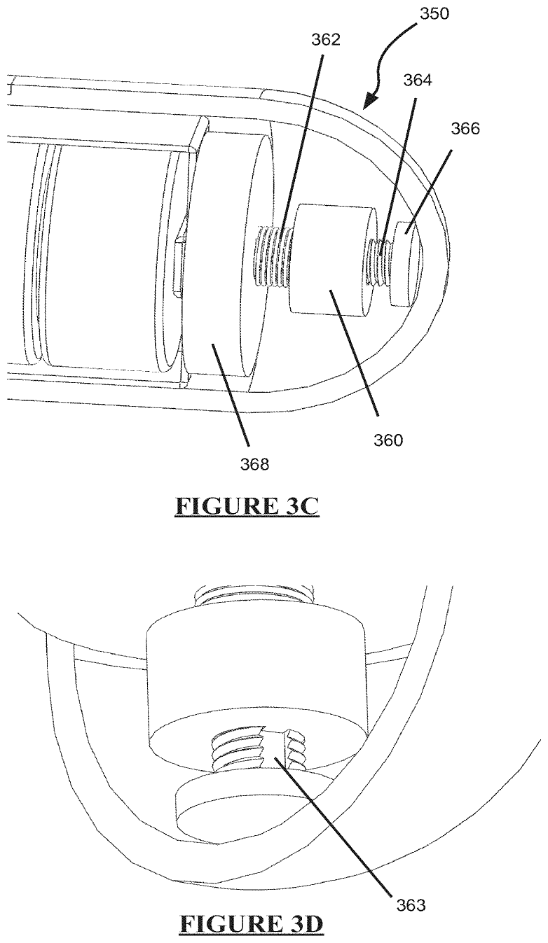

FIG. 3A is a schematic, top view of a cut-open GIC 300 according to some embodiments of the present invention. GIC 300 may include a capsule housing or shell 305 having complementary components 305A, 305B. Within capsule housing 305 may be disposed a thrusting mechanism that may include a motor 310 and a circuit board 322 having a CPU, microprocessor or controller 320. Within the capsule housing may further be disposed a power supply such as at least one battery 340, and an electrically conductive bridge such as metal bridge 330. An insulating barrier (shown in FIG. 1) may be disposed between battery 340 and bridge 330, to avoid short-circuiting.

As shown in FIG. 3A, two (by way of example) disc-shaped batteries 340 and circuit board 322 may be held together by metal bridge 330. Bridge 330 makes electrical contact with a broad face of the battery distal to circuit board 322, and may provide power to circuit board 322. Batteries 340 may also power motor 310, e.g., via conducting wires (not shown) attached to circuit board 322. Microprocessor or controller 320 may be mechanically and electrically attached to circuit board 322 by means of electrically conductive connectors 324.

As described hereinabove, the thrusting mechanism may be adapted to exert eccentric or radial forces on capsule housing 305. The motor may be an eccentric motor having an eccentric weight 312. As motor 310 spins in a generally normal fashion with respect to a longitudinal axis of GIC 300, radial forces are exerted on housing 305.

GIC 300 may be equipped with an auxiliary axial perturbation arrangement such as axial perturbation arrangement 350, adapted to effect axial forces on housing 305. The axial perturbation arrangement may be part of the thrusting mechanism. In the exemplary embodiment provided in FIG. 3A, perturbation arrangement 350 includes a motor 368 that is electrically connected to batteries 340. Motor 368 may be disposed at a distal end of GIC 300, with respect to motor 310.

Axial perturbation arrangement 350 may further include a motor screw or screw shaft such as axial motor screw shaft 364, mechanically associated with, and driven by, motor 368, and aligned in a generally axial fashion within GIC 300, typically along, generally along, or parallel to a longitudinal axis 308 of the capsule; a spring 362, which may be concentrically disposed on shaft 364, proximal to motor 310; a weight 360, which may be aligned in an axial fashion within GIC 300, and which may typically be disposed between spring 362 and motor screw 364; a stopper 366, adapted to receive a distal end (with respect to motor 310) of motor screw 364, and impinging against an inner wall 365 of capsule housing 305.

A side view of cut-open GIC 300 is provided in FIG. 3B.

FIG. 3C is a partial view of cut-open GIC 300, showing a magnified perspective view of perturbation arrangement 350, according to one embodiment of the invention.

FIG. 3D is another partial view of cut-open GIC 300, showing a magnified perspective view of an external interrupted thread 363 of screw shaft 364, according to one embodiment of the invention. Weight 360, which may be generally of an annular shape, may advantageously have a threaded internal surface (not shown) that may be generally complementary to the threading of interrupted thread 363. The interrupted portion of interrupted thread 363 may have a twin interrupted portion on the (radially and longitudinally) opposite side of screw shaft 364.

In one exemplary mode of operation of perturbation arrangement 350, screw shaft 364, driven by motor 368, engages the threaded internal surface of weight 360, such that weight 360 is drawn towards spring 362, and compression of spring 362 ensues (“State 1”). As screw shaft 364 continues to turn, the interrupted portion of interrupted thread 363 meets the threaded internal surface of weight 360, whereupon weight 360 becomes disengaged and longitudinally free with respect to interrupted thread 365. Spring 362, disposed in a compressed position, is now free to longitudinally extend (“State 2”), forcefully urging weight 360 towards stopper 366, and thereby axially impacting capsule housing 305. As screw shaft 364 continues to turn, screw shaft 364 again engages the threaded internal surface of weight 360, whereby perturbation arrangement 350 again reassumes State 1.

We have found that the ratio of the radial forces exerted to the axial forces exerted, on the housing, may be at least 1:1, at least 1.25:1, or at least 1.5:1, and more typically, at least 2:1, at least 3:1, at least 4:1, or at least 5:1.

Without wishing to be bound by theory, the inventor believes that the radial forces provide the primary effect of stimulating the walls of the lower gastrointestinal tract. Nonetheless, the axial forces may be useful in the locomotion of the capsule, particularly in regions that are partially clogged or blocked by chyme. Since the power supply is limited, a relatively high ratio of the radial forces exerted to the axial forces exerted may be critical in delivering the requisite stimulation to the walls of the tract.

The ratio of forces may be defined as the sum of the radial forces delivered to the sum of the axial forces delivered, over the entire time of activity of the GIC. For a GIC having a substantially repeating period, the ratio of forces may be defined as the sum of the radial forces delivered to the sum of the axial forces delivered, over one complete period.

In one embodiment, the GIC may be introduced to the body of the user via oral insertion.

In one embodiment, the GIC may be introduced into a lower end of the large intestine via the rectal opening. The general procedure may be similar to the introduction of a suppository. A first end of the GIC, which may have a tapered shape, and may be lubricated, may be placed at the rectal opening and gently pushed into the rectum. The GIC may be manually urged up the rectal tract, to a distance of several centimeters and up to about eight centimeters from the rectal opening. Deeper insertion, to the end of the rectum distal to the rectal opening, may be achieved by means of an insertion apparatus. Such an apparatus may include a long, smooth rod, preferably made of, or coated with, a flexible, smooth, biocompatible substance such as silicone. At a first end of the apparatus may be disposed a securing mechanism adapted to secure the GIC until the GIC has reached the desired position within the rectum, and a release mechanism adapted to release the GIC, upon demand. The securing and release mechanism may include a spring. Such an apparatus, whose structure or structures will be readily apparent to those of ordinary skill in the art, may enable the introduction of the GIC through the rectal tract, to a position of at least 8 cm, at least 10 cm, at least 12 cm, or at least 14 cm from the rectal opening.

In an actual capsule prototype, the capsule length was 24.2 mm, and the capsule diameter was 11.3 mm. The shell was made of medical Makrolon® 2458, a biocompatible material. The voltage was 4.5 Volts.

Following ingestion of the capsule, the vibrating sequence begins after a predetermined amount of time (delay). This delay (6 or 8 hours) may allow the capsule to reach the large intestine before the vibrating sequence is initiated.

The capsule may be activated by an electromagnetic signal carrying an activation code. The activation may be confirmed, e.g., by vibration of the capsule (e.g., 3 consecutive vibrations), or by any of various visual (e.g., LED) or audio signals, to ensure that the output (or the programming result) is identical to the requirements indicated by the physician.

The capsule typically contains an electromechanical system that operates a mechanically controlled vibrating mechanism adapted to induce peristaltic wave activity in the large intestine. A computerized algorithm may provide the vibration rate and relaxation period in order to prevent habituation.

Various therapeutic modes may be pre-programmed or pre-set for the GIC. For example:

Mode A: activation delay is set to 8 hours. The vibration rate is 180 vibration cycles per hour, each cycle consisting of 4 seconds of a vibration period and 16 seconds of a repose (relaxation) period, corresponding, on a per hour basis, to 12 minutes of vibration periods and 48 minutes of rest intervals or periods.

Mode B: activation delay is set to 6 hours. The vibration rate is 240 vibration cycles per hour, each cycle consisting of 4 seconds of a vibration period and 11 seconds of a repose period, corresponding, on a per hour basis, to 16 minutes of vibration periods and 44 minutes of rest intervals or periods.

To ensure that the capsule has reached the large intestine, the capsule is equipped with an activation delay mechanism (typically having a pre-determined delay of 6-8 hours) that defines the time period between activation (and typically, ingestion) and the initial onset of the vibrating phase.

The capsule may be advantageously activated by qualified medical personnel. In some cases, the capsule may be activated by the user.

In some embodiments of the present invention, various dedicated GI capsules may be produced, that may be pre-programmed according to the needs of various patients. Such embodiments may not require the transmitter and antenna.

In some embodiments employing programming according to the needs of the patient:

A. The capsule may be equipped with an electronic circuit, transmitter and antenna, adapted to receive an external signal regarding the mode of activation required.

B. The capsule may be activated via a dedicated base unit. The base unit may include an electronic circuit, a power supply (batteries), software and a socket adapted to receive the capsule. The base unit has various programming modes that enable the medical personnel to select the appropriate one according to the specific needs of the patient/user, e.g., according to the severity of the constipation (e.g., Rome II, Rome III, etc.).

C. The activation of the capsule with the selected mode of operation is performed by the dedicated base unit, which may transmit to the capsule the programmed mode, by a simple push of a button on the base unit.

D. The capsule will signal that it received the mode of work chosen, and after the signal, it is activated and ready to be swallowed.

As mentioned above, we have discovered a method for reducing a straining sensation of a human subject during defecating and increasing Bristol stool scores of fecal matter of the subject using an ingestible vibrating gastrointestinal capsule. We have found that when a human subject, suffering from a straining sensation during defecating and/or having fecal matter which is low on a Bristol stool scale, ingests a vibrating gastrointestinal capsule according to a particular treatment schedule, and the capsule vibrates within the gastrointestinal tract of the subject in accordance with a vibration protocol, the straining sensation is reduced or alleviated and the Bristol stool score improves.

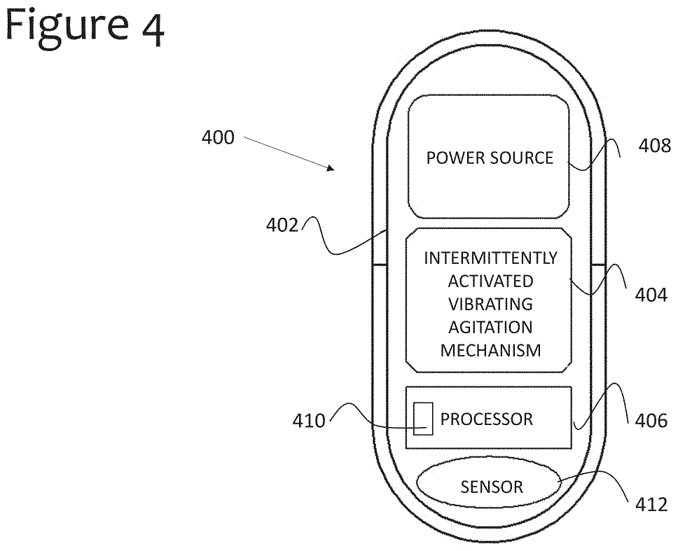

As seen in FIG. 4, vibrating ingestible capsule 400 includes a capsule housing or shell 402, having disposed therein a vibrating agitation mechanism 404 adapted to be intermittently activated, a processor 406 adapted to control operation of vibrating agitation mechanism 404, and at least one power source 408 providing power to vibrating agitation mechanism 404 and processor 406. In some embodiments, the processor is functionally associated with a timer 410. In some embodiments, capsule 400 further includes at least one sensor 412, functionally associated with processor 406. The at least one sensor 412 may be adapted to sense at least one parameter within capsule 400 or in an environment of capsule 400, and may include a temperature sensor, a moisture sensor, a pH sensor, or any other suitable sensor.

Power source 408 may be any suitable power source, such as, for example, one or more alkaline or silver oxide batteries, primary batteries, rechargeable batteries, capacitors and/or super capacitors.

Intermittently activated vibrating agitation mechanism 404 is adapted to have a vibration mode of operation and a rest mode of operation. In the vibration mode of operation, intermittently activated vibrating agitation mechanism 404 is adapted to exert forces on capsule housing 402, such that capsule housing 402 exerts vibrations on an environment surrounding capsule 400.

In some embodiments, intermittently activated vibrating agitation mechanism 404 may include a radial agitation mechanism adapted to exert radial forces on the capsule housing 402, in a radial direction with respect to a longitudinal axis of housing 402. For example, the radial agitation mechanism may include an unbalanced weight attached to a shaft of an electric motor powered by said battery, substantially as described in U.S. Pat. No. 9,707,150, which is incorporated by reference for all purposes as if fully set forth herein.

In some embodiments, the intermittently activated vibrating agitation mechanism 404 may include an axial agitation mechanism adapted to exert radial forces on the capsule housing 402, in an axial direction with respect to a longitudinal axis of housing 402. For example, the axial agitation mechanism may include an electric motor powered by the battery and an urging mechanism, associated with, and driven by, the electric motor, such that the urging mechanism adapted to exert said axial forces, substantially as described in U.S. Pat. No. 9,707,150. In some embodiments, the urging mechanism adapted to exert the axial forces in opposite directions. In some embodiments, the urging mechanism is adapted to deliver at least a portion of the axial forces in a knocking mode.

In some embodiments, the forces exerted by intermittently activated vibrating agitation mechanism 404 on capsule housing 402 in the vibration mode of operation include radial forces in a radial direction with respect to the longitudinal axis of the housing and axial forces in an axial direction with respect to the longitudinal axis. In some embodiments, a single agitation mechanism exerts both the radial and the axial forces. In other embodiments, the axial forces are exerted by one agitation mechanism, and the radial forces are exerted by another, separate, agitation mechanism, where both agitation mechanisms form part of intermittently activated vibrating agitation mechanism 404.

In the vibrating mode of operation, intermittently activated vibrating agitation mechanism 404 is adapted to have a plurality of vibration cycles, where each cycle includes a vibration duration followed by a repose duration. Forces are exerted by vibrating agitation mechanism 404 on capsule housing 402 only during the vibration duration, and as such capsule housing 402 only exerts forces on an environment thereof during the vibration duration.

In some embodiments, the number of vibration cycles per hour is in the range of 20 to 400, 40 to 400, 60 to 400, 80 to 400, 40 to 380, 60 to 380, 80 to 380, 40 to 360, 60 to 360, 80 to 360, 100 to 360, 100 to 330, 100 to 300, 100 to 280, 100 to 250, 100 to 220, 100 to 200, 120 to 300, 120 to 280, 120 to 250, 120 to 220, 120 to 200, 150 to 300, 150 to 280, 150 to 250, 150 to 220, 150 to 200, 170 to 300, 170 to 250, 170 to 220, or 170 to 200.

In some embodiments, the repose duration is greater than the vibration duration.

In some embodiments, the vibration duration is in the range of 0.1 second to 10 seconds, 1 second to 10 seconds, 1 second to 9 seconds, 2 seconds to 9 seconds, 3 seconds to 9 seconds, 3 seconds to 8 seconds, 3 seconds to 7 seconds, 4 seconds to 6 seconds, or 5 seconds to 6 seconds.

In some embodiments, the repose duration is in the range of 1 second to 180 seconds, 3 seconds to 180 seconds, 5 seconds to 180 seconds, 5 seconds to 150 seconds, 5 seconds to 120 seconds, 8 seconds to 100 seconds, 8 seconds to 30 seconds, 10 seconds to 80 seconds, 10 seconds to 70 seconds, 10 seconds to 60 seconds, 10 seconds to 50 seconds, 10 seconds to 40 seconds, 10 seconds to 30 seconds, 10 seconds to 20 seconds, or 15 seconds to 20 seconds.

In some embodiments, the total duration of one vibration cycle is in the range of 1.1 seconds to 200 seconds, 5 seconds to 200 seconds, 10 seconds to 200 seconds, 10 seconds to 150 seconds, 10 seconds to 100 seconds, 10 seconds to 80 seconds, 10 seconds to 50 seconds, 10 seconds to 40 seconds, 10 seconds to 30 seconds, 15 seconds to 50 seconds, 15 seconds to 40 seconds, 15 seconds to 30 seconds, or 15 seconds to 25 seconds.

In some embodiments, the cumulative duration of the vibrating mode of operation, or the cumulative duration during which vibration cycles are occurring, is in the range of 1 hour to 12 hours, 2 hours to 10 hours, 2 hours to 8 hours, 2 hours to 6 hours, 2 hours to 4 hours, or 2 hours to 3 hours. It will be appreciated that the cumulative duration of vibration cycles may be dependent on properties of power source 408.

It will be appreciated by persons skilled in the art that the vibration mode of operation may be intermittent, or interrupted, such that vibrating agitation mechanism 404 is operative in the vibration mode for a first duration, for example 30 minutes, then does not vibrate, or have any vibration cycles, for a second duration, for example 1 hour, and then is operative in the vibration mode and has vibration cycles for a third duration, for example two hours. The cumulative duration relates to the sum of all durations during which the vibrating agitation mechanism 404 was operative in the vibration mode and included vibration cycles, including the vibration duration and the repose duration of these vibration cycles.

In some embodiments, vibrating agitation mechanism 404 is configured to exert forces on capsule housing 402, such that a net force exerted by capsule housing 402 on the environment thereof is in the range of 50 grams force (gf) to 600 gf, 50 gf to 550 gf, 100 gf to 550 gf, 100 gf to 500 gf, 150 gf to 500 gf, 200 gf to 500 gf, or 200 gf to 450 gf.

In some embodiments, vibrating agitation mechanism 404 is configured to exert said forces on capsule housing 402 to attain a capsule housing 402 vibrational frequency within a range of 10 Hz to 650 Hz, 15 Hz to 600 Hz, 20 Hz to 550 Hz, 30 Hz to 550 Hz, 50 Hz to 500 Hz, 70 Hz to 500 Hz, 100 Hz to 500 Hz, 130 Hz to 500 Hz, or 150 Hz to 500 Hz.

It will be appreciated that the exact specifications of the capsule, such as the specific frequency and force ranges applicable to a specific capsule, are dependent on the specifications of the power source and of the vibrating agitation mechanism.

It will be further appreciated that a specific capsule may be controlled by the processor such that different vibrational frequencies may be attained and/or different net forces may be exerted, by the capsule in different vibration cycles of the capsule. Due to the natural distinction between subjects, use of multiple different parameters in different vibration cycles of a single capsule would allow the capsule to successfully treat multiple subjects, even if the personal optimal treatment for those subjects is not the same, as there is a higher chance that in at least some of the vibration cycles the activation parameters of the capsule would reach, or be close to, the optimal parameters for each specific subject.

Processor 406 is adapted to control the operation of intermittently activated vibrating agitation mechanism 404. Such control may include control of any one or more of the force applied by the vibrating agitation mechanism, the vibrational frequency reached, the times in which vibrating agitation mechanism 404 operates in the vibration mode of operation, the vibration duration of each vibration cycle, the repose duration of each vibration cycle, the vibration cycle duration, and cumulative vibration duration of the vibrating agitation mechanisms.

In some embodiments, processor 406 is adapted to wait for a pre-set activation time delay following activation of capsule 400 and prior to initiation of the vibration mode of operation of vibration agitation mechanism 404. The activation time delay may be any suitable time delay, and may be dependent on portions of the gastrointestinal tract in which it is desired that the capsule will operate.

For example, in embodiments in which it is desired that the capsule operate, or vibrate, in an intestinal portion, the activation time delay may be in the range of 2 hours to 48 hours, 2 hours to 42 hours, 2 hours to 36 hours, 2 hours to 30 hours, 2 hours to 24 hours, 3 hours to 24 hours, 4 hours to 24 hours, 4 hours to 20 hours, 4 hours to 18 hours, 4 hours to 16 hours, 4 hours to 14 hours, 4 hours to 12 hours, 6 hours to 12 hours, or 6 hours to 10 hours.

In some embodiments, processor 406 is adapted to receive information relating to the desired vibration protocol from a control unit (not shown), prior to ingestion of the capsule or to activation thereof. For example, the information may be remotely transmitted from the control unit to processor 406, for example using a short range wireless communication method. In some embodiments, the information is transmitted as a list of vibration parameters for effecting the vibration protocol. In some embodiments, the information is transmitted as executable code for effecting the first vibration protocol.

In some embodiments, the information includes one or more of a desired activation time delay, a desired number of vibration cycles, a desired vibration duration in each vibration cycle, a desired repose duration in each vibration cycle, a desired cumulative vibration duration, and the like.

In some embodiments, processor 406, or a timer associated therewith, is adapted to be activated by the control unit prior to ingestion of capsule 400. In some embodiments, activation is carried out by sending a signal to processor 406, for example using a short range wireless communication protocol. In some embodiments, the activation signal activates the timer to immediately begin effecting the vibration protocol. In some embodiments, at least one sensor 408 is adapted to identify ingestion of the capsule, and processor 406 is adapted to begin effecting the vibration protocol immediately following identification of ingestion of capsule 400.

In some embodiments, processor 406 is adapted to control vibrating agitation mechanism 404 so that the capsule applies forces to an environment thereof to effect a mechanical stimulation of the wall of the gastrointestinal tract of the subject in a targeted zone.

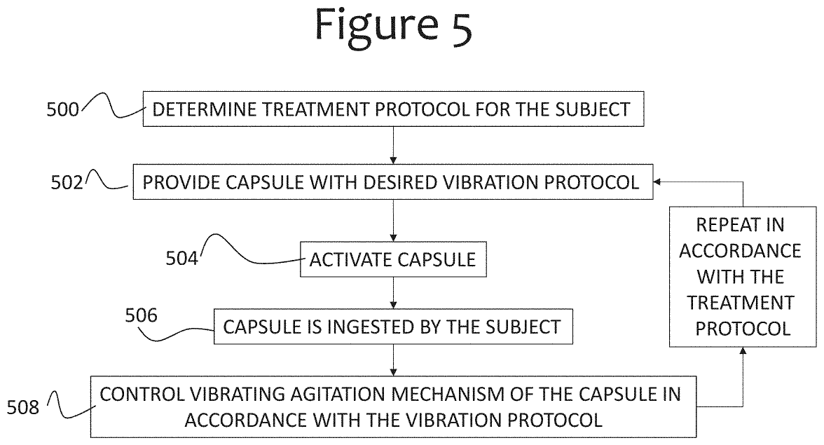

Reference is now additionally made to FIG. 5, which is a schematic flowchart of a method of treatment for reducing a straining sensation of a human subject during defecating and improving Bristol stool scores of fecal matter of the subject according to the present invention, the treatment being based one use of an ingestible vibrating gastrointestinal capsule, such as capsule 400 of FIG. 4.

As seen at step 500, initially the treatment protocol for the subject is set or determined, for example by a treating physician or medical practitioner. The treatment protocol may indicate the number of treatment sessions per week or per other time duration, the time of day at which a capsule should be ingested, a targeted zone in which the capsule should be operative, and/or may indicate the vibration protocol of the capsule.

At step 502, the processor 406 of an ingestible capsule 400 may optionally receive, or be programmed with, a desired vibration protocol, in accordance with the treatment protocol determined at step 500. In some embodiments, such programming of the desired vibration protocol is effected by a control unit. For example, the programming may include remotely transmitting the desired vibration protocol from the control unit to processor 406, for example using a short range wireless communication method. In some embodiments, the desired vibration protocol is transmitted as a list of vibration parameters for effecting the vibration protocol. In some embodiments, the desired vibration protocol is transmitted as executable code for effecting the vibration protocol.

In some embodiments, step 502 includes pre-setting of an activation time delay for activation of the capsule. In some embodiments, However, in some embodiments, ingestible capsule 400 may be pre-programmed, for example with a default vibration protocol or with a pre-set protocol, in which case step 502 may have been previously executed, e.g., by the capsule manufacturer.

The vibration protocol pre-set or programmed into capsule 400, and specifically the activation time delay of the capsule, is selected to effect vibration of capsule 400 when the capsule will be located in a targeted zone within the gastrointestinal tract of the subject. In some embodiments, the targeted zone is defined in the treatment protocol determined at step 500. In some embodiments, the targeted zone is an intestinal section of the gastrointestinal tract of the subject. In some such embodiments, the targeted zone is in a section of the large intestine or the rectum of the subject.

In some embodiments, in which the targeted zone includes an intestinal section of gastrointestinal tract, the activation time delay is selected to be in the range of 2 hours to 48 hours, 2 hours to 42 hours, 2 hours to 36 hours, 2 hours to 30 hours, 2 hours to 24 hours, 3 hours to 24 hours, 4 hours to 24 hours, 4 hours to 20 hours, 4 hours to 18 hours, 4 hours to 16 hours, 4 hours to 14 hours, 4 hours to 12 hours, 6 hours to 12 hours, or 6 hours to 10 hours.

In some embodiments, the selected activation time delay is selected according to a measured or estimated transit time of chyme along the gastrointestinal tract of the subject being treated. In some such embodiments, information relating to the measured or estimated transit time of chyme is collected prior to step 502.

The capsule is activated for use at step 504. In some embodiments, activation is performed automatically when the capsule receives the vibration protocol, at step 502. In other embodiments, such as in embodiments in which the vibration protocol is pre-set, the capsule may be explicitly activated, such as by receipt of an activation signal from the control unit or by sensors within the capsule sensing that the capsule has been ingested. Activation of the capsule results in activation of the timer associated with the processor 406, and is the start of the activation time delay.

Following activation of capsule 400, or together therewith, capsule 400 is ingested by the subject, and begins to travel through the gastrointestinal tract of the subject, as seen at step 506.

At step 508, while capsule 400 is travelling in the gastrointestinal tract together with the food/chyme therein, processor 406 controls vibrating agitation mechanism 404 in accordance with the vibration protocol, so that vibrating agitation mechanism 404 is in the vibrating mode of operation when the capsule is disposed in the targeted zone.

Operation of vibrating agitation mechanism 404 in the vibrating mode of operation effects vibration of capsule housing 402, as described hereinabove, such that the housing exerts vibrations on the environment surrounding the capsule in the targeted zone. Specifically, vibration of capsule housing 402 may be intended to effect a mechanical stimulation of the wall of the gastrointestinal tract in the targeted zone.

A treatment session as defined in steps 502 to 508 may be repeatedly administered to the subject as specified in the treatment protocol for the subject, determined or obtained at step 500. In some embodiments, the treatment protocol includes administering a plurality of treatment sessions to the subject. In some embodiments, the treatment protocol includes administering at least one treatment session to the subject per week, over a treatment period of at least two weeks, at least at least three weeks, at least four weeks, at least five weeks, at least six weeks, or at least eight weeks. In some embodiments, the treatment protocol includes administering 1 to 7 treatment sessions per week, 3 to 14 treatment sessions per two weeks, 2 to 7 treatment sessions per week, 5 to 14 treatment sessions per two weeks, 3 to 7 treatment sessions per week, 7 to 14 treatment sessions per two weeks, 4 to 7 treatment sessions per week, or 5 to 7 treatment sessions per week.

The subject may be any suitable subject, suffering from a sensation of straining during defecating and/or whose fecal matter has a low score on the Bristol stool scale. The sensation of straining during defecating and/or a low score on the Bristol stool scale may be caused by any of a number of underlying conditions, such as chronic constipation, food allergies or intolerances, hormonal or enzymatic deficiencies, irritable bowel syndrome, colon cancer, medications taken by the subject which change the consistency of the fecal matter, neurological diseases, and hypothyroidism.

In some embodiments, the subject is a subject who has experienced at most one of the following symptoms over the 3 months preceding the beginning of treatment:

fewer than three bowel movements per week;

straining;

lumpy or hard stools;

sensation of anorectal obstruction;

sensation of incomplete defecation; and

manual maneuvering required to defecate.

In some embodiments, the subject is a subject who has experienced at most two of the following symptoms over the 3 months preceding the beginning of treatment:

fewer than three bowel movements per week;

straining;

lumpy or hard stools;

sensation of anorectal obstruction;

sensation of incomplete defecation; and

manual maneuvering required to defecate.

In some embodiments, the subject is a subject who has experienced at most one of the following symptoms over the 3 months preceding the beginning of treatment:

fewer than three bowel movements per week;

straining during more than 25% of defecations;

lumpy or hard stools in more than 25% of defecations;

sensation of incomplete defecation in more than 25% of defecations;

sensation of anorectal obstruction in more than 25% of defecations; and

manual maneuvering required to facilitate more 25% of defecations.

In some embodiments, the subject is a constipation free subject.

In some embodiments, the subject suffers from emotional stress and/or from psychosomatically caused symptoms.

### EXAMPLES

Reference is now made to the following examples, which together with the above descriptions, illustrate the invention in a non-limiting fashion.

### Example 1

Clinical trials on patients were performed using the GIC described with reference to FIGS. 3 and 4. A synopsis of the study protocol is provided in Appendix I.

After an initial, two-week baseline period, in which the number of spontaneous bowel movements was recorded, the GIC was administered about twice per week for a period of close to 7 weeks.

The activation delay of the capsules was set to 8 hours. The vibration rate was 180 vibration cycles per hour, each cycle consisting of 4 seconds of a vibration period and 16 seconds of a repose period. The vibration frequency was about 27 Hz. The average force exerted by the vibrations was 64 gf, while the maximal (instantaneous) force exerted was about 176 gf.

Following the activation delay, the therapeutic treatment was conducted for about 2-2.5 hours.

Efficacy was assessed by the increase in spontaneous bowel movement per week during the 7 weeks of treatment, as compared to a two-week baseline period. The efficacy assessment was performed for the Per Protocol population.

An increase in the mean number of spontaneous bowel movements per week was observed (see Table 1). This increase was found to be statistically significant (Mean increase=1.7, Standard deviation=1.1, p<0.001).

Following the study, the status of the patients was monitored for a period of 6 months. With regard to constipation, it was found that after this six-month period, over 40% of the patients continued to enjoy an improved situation, while the situation of about 90% of the patients was better or unchanged.

TABLE 1

| Patient | 2 week | Treatment | | |
|---|---|---|---|---|
| No. | baseline | period | Change | Change (%) |
| 1 | 2.5 | 5.4 | 2.9 | 116% |
| 2 | 2.0 | 2.3 | 0.3 | 15% |
| 3 | 1.0 | 3.4 | 2.4 | 240% |
| 4 | 2.0 | 4.5 | 2.5 | 125% |
| 5 | 2.5 | 3.5 | 1.0 | 40% |
| 6 | 2.5 | 2.3 | −0.2 | −8% |
| 7 | 1.5 | 3.5 | 2.0 | 133% |
| 8 | 2.5 | 2.6 | 0.1 | 4% |
| 9 | 4.0 | 6.3 | 2.3 | 57% |
| 10 | 2.0 | 2.6 | 0.6 | 30% |
| 11 | 3.0 | 5.4 | 2.4 | 80% |
| 12 | 2.5 | 5.0 | 2.5 | 100% |
| 13 | 2.0 | 4.4 | 2.4 | 120% |
| 14 | 2.0 | 4.8 | 2.8 | 140% |

TABLE 2A

| Patients status after 6 | | |
|---|---|---|
| months | N | % |
| Worse or No change | 6 | 54.5 |
| Better | 5 | 45.5 |

TABLE 2B

| | Value |
|---|---|
| Proportion | 0.455 |
| 95% Lower Conf | 0.167 |
| Limit | |
| 95% Upper Conf | 0.766 |
| Limit | |

TABLE 3A

| Patients status after 6 | | |
|---|---|---|
| months | N | % |
| Worse | 1 | 9.1 |
| Better or No change | 10 | 90.9 |

TABLE 3B

| | Value |
|---|---|
| Proportion | 0.909 |
| 95% Lower Conf | 0.587 |
| Limit | |
| 95% Upper Conf | 0.998 |
| Limit | |

The GICs of the present invention are effective in treating various levels of constipation, including Rome I, Rome II and Rome III levels. The GICs of the present invention may be effective in treating more serious levels of constipation, including Rome IV, Rome V and Rome VI levels.

The GICs of the present invention have been found to be effective in relieving constipation accompanied by abdominal pain.

According to the Rome III criteria for constipation, by way of example, a patient must have experienced at least 2 of the following symptoms over the preceding 3 months:
Fewer than 3 bowel movements per week
Straining
Lumpy or hard stools
Sensation of anorectal obstruction
Sensation of incomplete defecation
Manual maneuvering required to defecate.

Thus, according to one aspect of the present invention there is provided a method for mechanically stimulating a wall of a segment of a mammalian gastrointestinal tract of a user by means of a gastrointestinal capsule, the method including the steps of: (a) providing at least one capsule (preferably any one of the capsules disclosed herein); and (b) administering at least one treatment session, each treatment session including: (i) delivering the gastrointestinal capsule into the tract; and (ii) effecting activation of a thrusting mechanism of the gastrointestinal capsule to achieve mechanical stimulation of the wall of the gastrointestinal tract.

The at least one treatment session may advantageously include a plurality of treatment sessions. Typically, at least one of the treatment sessions is administered per week, over a treatment period extending for at least two weeks, at least three weeks, at least four weeks, at least five weeks, at least six weeks, or at least eight weeks. At least 1.5, at least 1.75, at least 2, at least 2.5, or at least 3 of the treatment sessions may be administered per week of the treatment period.

In some embodiments, the frequency of the treatment sessions administered to the user is within a range of 1.5 to 6 per week of the treatment period.

In some embodiments, the frequency is within a range of 1.5 to 5, 1.5 to 4, 1.5 to 3.5, 1.5 to 3, 2 to 6, 2 to 5, 2 to 4, 2 to 3.5, or 2 to 3, per week of the treatment period.

In some embodiments, within each treatment session, the activation of the thrusting mechanism is performed for a duration effective to achieve mechanical stimulation of the wall of the gastrointestinal tract.

In some embodiments, within each treatment session, the activation of the thrusting mechanism is performed for a duration effective to increase a frequency of spontaneous bowel movements of the user.

In some embodiments, within each treatment session, the activation of the thrusting mechanism is performed for a duration effective to increase a frequency of spontaneous bowel movements of the user by at least 25%, at least 50%, at least 75%, or at least 100%.

In some embodiments, within each treatment session, the activation of the thrusting mechanism is performed is performed for a duration effective to at least partially relieve a condition of constipation of the user.

In some embodiments, within each treatment session, the activation of the thrusting mechanism is performed for a duration effective to completely relieve a condition of constipation of the user.

In one embodiment of the invention, the gastrointestinal capsule includes a housing; a thrusting mechanism, disposed within the housing, having an active mode and a mode that is passive with respect to the active mode. The thrusting mechanism may be adapted to exert a radial force on the housing, whereby, when the capsule is disposed within a gastrointestinal tract of a user, and the mechanism is in the active mode, the gastrointestinal capsule stimulates the walls of the tract. This active mode may advantageously include a series of at least two pulses of such radial force, the series characterized by a first duration. The passive mode may be characterized by a second duration that exceeds the first duration.

We have found that for a GIC activation cycle defined by the active mode followed by the passive mode (or, the series of pulses of radial force immediately followed by the second duration of the passive mode), the period of the activation cycle may advantageously be within a range of 5-60 seconds.

According to some embodiments of the invention, the activation cycle period may be at least 5, at least 6, at least 7, at least 8, at least 10, at least 12, or at least 15, seconds.

According to some embodiments of the invention, the activation cycle period may be at most 60, at most 40, at most 30, at most 25, or at most 20 seconds.

According to some embodiments of the invention, the first duration of the active mode of the activation cycle period may be within a range of 1-10 seconds.

According to some embodiments of the invention, this first duration may be within a range of 2-8 seconds.

According to some embodiments of the invention, this first duration may be within a range of 2.5-6 seconds.

According to some embodiments of the invention, the thrusting mechanism of the capsule is designed and adapted to produce a vibrational frequency within a range of 12 Hz to 80 Hz, within a range of 12 Hz to 70 Hz, within a range of 15 Hz to 60 Hz, within a range of 15 Hz to 50 Hz, or within a range of 18 Hz to 45 Hz. We have found that within these narrow ranges of vibrational frequencies, the GIC exhibits superior performance in treating gastrointestinal disorders, and more particularly, constipation and the like.

We have further discovered that the magnitude of the net force exerted by the capsule on its surroundings within the gastrointestinal tract may be pivotal in the efficacy of the GIC. According to an embodiment of the present invention, the magnitude of the net force on an environment external to the GIC may be at least 400 grams force, at least 450 grams force, at least 500 grams force, or at least 600 grams force.

### Example 3

A study was conducted, in which 24 participating subject suffering from a sensation of straining while defecating and whose fecal matter had a Bristol stool score of 1-2 were treated with a vibrating gastrointestinal capsule according to a treatment protocol.

The treatment protocol included treatment cycles including administering one vibrating gastrointestinal capsule per day for two days, followed by one day where no capsule is administered, repeated for a treatment duration of six weeks.

At the end of each week of treatment, as well as at the end of a two week run-in period preceding the initiation of treatment, the subjects were asked to rank the degree to which they felt a sensation of straining during defecating, on a scale of 1 to 11, where 11 represents a severe straining sensation and 1 represents a very mild or infrequent straining sensation. Additionally, at the beginning of the run-in period, at the beginning of the treatment, at the midpoint of the treatment, and at the end of the treatment, the subjects were asked to indicate the Bristol stool score of fecal matter thereof, based on a Bristol stool chart.

The administered capsules included a zinc-manganese dioxide alkaline battery, such as a AG3/LR41 button cell, commercially available from Daly-Station Battery Limited of Shenzhen Guandong, P.R. China, as the power source, and a coin type eccentric vibration motor, such as a coin-type motor having the Product Part No. C0834L-066332017-2001 commercially available from Ineed HK Limited of Kowloon, Hong-Kong, as the vibrating agitation mechanism.

The administered capsules were programmed to have a activation time delay of 8 hours, and, when in the vibration mode of operation, to have vibration treatment cycles including a 3 second vibration duration followed by a 16 second repose duration, for a cumulative treatment duration of 2.5 to 3 hours. The force applied by the capsule housing to the environment therearound during the vibration mode of operation was in the range of 200 gram-force to 500 gram-force, and the vibrational frequency was in the range of 120 Hz to 250 Hz. Different specific forces were applied to the surrounding environment, and corresponding different vibrational frequencies were attained, in different vibration cycles of the administered capsules.

Due to the activation time delay, it is assumed that vibration was affected when the capsules were disposed in a section of the large intestine of the participating subjects.

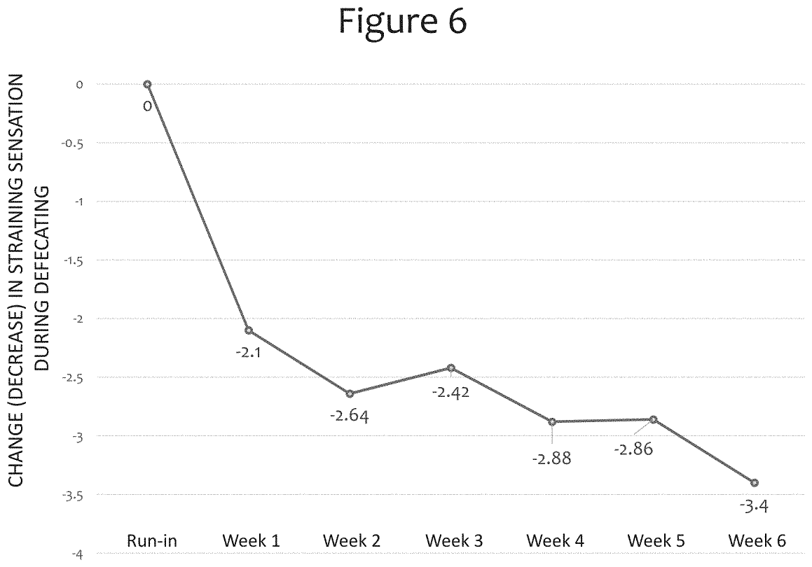

The results of the study with respect to the sensation of straining during defecating are shown in FIG. 6, which illustrates the improvement in the average degree of straining sensed by the participating subjects vs. treatment time. As seen in FIG. 6, the end of the run-in period sets the baseline measure for the average degree of straining sensed by participants, and as such shows 0 improvement relative to baseline. During the treatment period, the average degree of straining sensed by participants appears to monotonically improve, until at the end of the 6-week treatment period, =the average degree of straining during defecating sensed by the participants was more than three rankings lower, indicating a significant improvement in the degree of straining during defecating sensed by the participants. As such, the results illustrated in FIG. 6 are indicative of the success of the treatment of the present invention in reducing the sensation of straining during defecating.

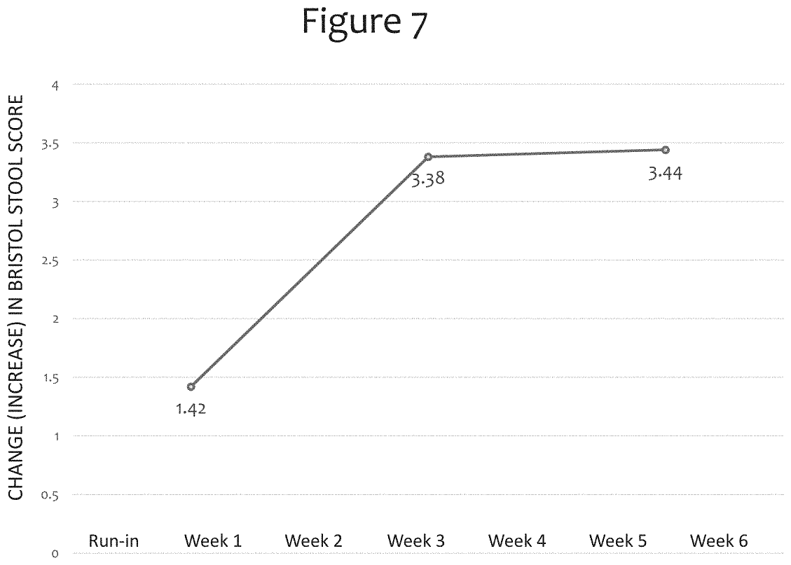

The results of the study with respect to a Bristol stool score of fecal matter are shown in FIG. 7, which illustrates the average Bristol stool score indicated by the participating subjects vs. treatment time. As seen in FIG. 7, at the beginning of treatment, the average Bristol stool score was less than 1.5, indicative of hard stool which is difficult to pass. An improvement was felt by the participants by the trial midpoint, after three weeks of treatment, when the average Bristol stool score was 3.38, indicative of normal stool. The average Bristol stool score remained within the normal range (between 3 and 4) until the end of the treatment period, after 6 weeks. As such, the results illustrated in FIG. 7 are indicative of the success of the treatment of the present invention in increasing the Bristol stool score of fecal matter by 2 points on a 7 points scale, for subjects treated by the method of FIG. 5, and specifically, that fecal matter of such subjects, on average, moved from being hard to pass to being normal.

### Example 4

A study which included 150 participating subjects suffering from a sensation of straining while defecating and whose fecal matter had a Bristol stool score of 1-2 was conducted. Half of the participating subjects, termed herein “trial subjects”, were treated with a vibrating gastrointestinal capsule according to a treatment protocol, in accordance with the present invention, while the other half, termed herein “sham subjects”, were treated with a sham capsule, which appeared and behaved identically to the vibrating gastrointestinal capsule prior to ingesting thereof, but did not vibrate within the subject's alimentary tract.

The treatment protocol included treatment cycles including administering one capsule per day for two days, followed by one day where no capsule is administered, repeated for a treatment duration of six weeks, where the trial subjects received a vibrating gastrointestinal capsule, and the sham subjects received a sham capsule.

At the end of each week of treatment, as well as at the end of a two week run-in period preceding the initiation of treatment, the subjects were asked to rank the degree to which they felt a sensation of straining during defecating, on a scale of 1 to 11, where 11 represents a severe straining sensation and 1 represents a very mild or infrequent straining sensation. Additionally, at the beginning of the run-in period, at the beginning of the treatment, at the midpoint of the treatment, and at the end of the treatment, the subjects were asked to indicate the Bristol stool score of fecal matter thereof, based on a Bristol stool chart.

The administered capsules included a zinc-manganese dioxide alkaline battery, such as a AG3/LR41 button cell, commercially available from Daly-Station Battery Limited of Shenzhen Guandong, P.R. China, as the power source, and a coin-type eccentric vibration motor, such as a coin-type motor having the Product Part No. C0834L-066332017-2001, commercially available from Ineed HK Limited of Kowloon, Hong-Kong, as the vibrating agitation mechanism.

The capsules administered to the trial subjects were programmed to have a activation time delay of 8 hours, and, when in the vibration mode of operation, to have vibration treatment cycles including a 3 second vibration duration followed by a 16 second repose duration, for a cumulative treatment duration of 2.5 to 3 hours. During the vibration mode of operation, the force applied by the capsule housing on the surrounding environment was in the range of 200 gram-force to 500 gram-force, and the vibrational frequency was in the range of 120 Hz to 250 Hz. Different specific forces were applied to the surrounding environment, and corresponding different vibrational frequencies were attained, in different vibration cycles of the administered capsules.

Due to the activation time delay, it is assumed that vibration was affected when the capsules were disposed in a section of the large intestine of the participating subjects.

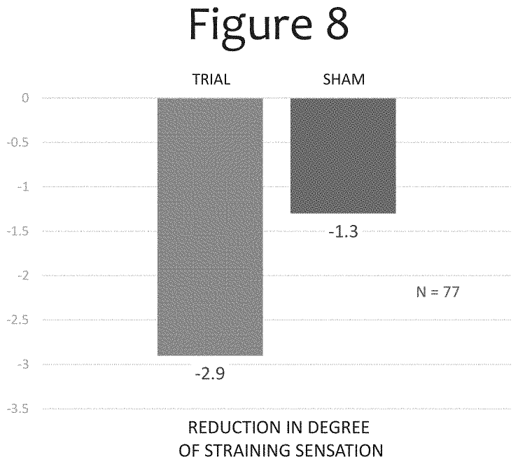

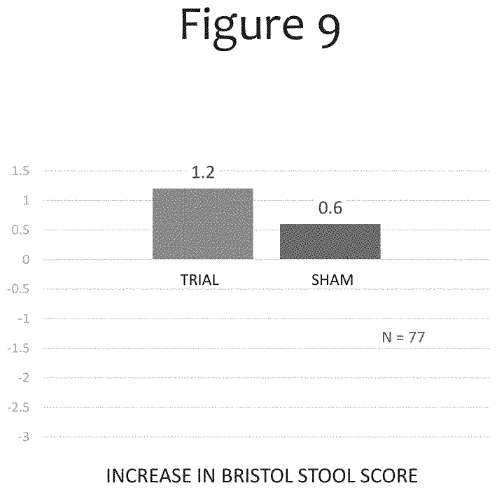

FIGS. 8 and 9 illustrates the improvement in the average degree of straining sensed by the participating subjects at the end of the study and the improvement in the average Bristol stool score for 50% of the participating subjects having moderate to severe symptoms, and removing subjects suffering from mild symptoms and from verey severe symptoms. Stated differently, the results illustrated in FIGS. 8 and 9 relate to the 50% of the participants at the center of the Gaussian curve of symptom severity for both the trial subjects and the sham subjects.

As seen in FIG. 8, at the end of the 6-week treatment period, the average degree of straining during defecating sensed by the trial subjects having moderate to severe symptoms was almost three whole rankings (2.9 rankings) lower, indicating a significant improvement in the degree of straining during defecating sensed by those subjects. By contrast, the average degree of straining during defecating sensed by the sham subjects was 1.3 ranking lower. As such, the results illustrated in FIG. 8 indicate that for a statistically-significant subject pool, (i) the inventive treatment method appreciably alleviated straining sensations during defecating, and (ii) the favorable results are well above and beyond the more minor degree of alleviation associated with placebo effects.

As seen in FIG. 9, at the end of the 6-week treatment period, the average Bristol stool score of fecal matter as sensed by the trial subjects having moderate to severe symptoms was 1.2 rankings higher, indicating a significant improvement in the Bristol stool score of fecal matter of those subjects, who, at the start of the trial, had very low Bristol stool scores. By contrast, the average Bristol stool score of fecal matter as sensed by the sham subjects was, on average, 0.6 rankings higher. As such, the results illustrated in FIG. 9 indicate that for a statistically-significant subject pool, (i) the inventive treatment method appreciably increases the Bristol stool score of subjects initially having a low Bristol stool score, and (ii) the favorable results are well above and beyond the more minor degree of improvement associated with placebo effects.

It will be appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A method of treating a sensation of straining while defecating in a human subject using a gastrointestinal capsule adapted to transit an alimentary canal of the subject, said capsule having:
a housing;
a battery, disposed within said housing; and
a vibrating agitation mechanism, powered by said battery, said vibrating agitation mechanism adapted such that, in a first vibrating mode of operation, said housing exerts vibrations on an environment surrounding said capsule, the method comprising:
(a) ingesting said gastrointestinal capsule; and
(b) controlling said vibrating agitation mechanism such that said first vibrating mode of operation occurs when said capsule is disposed within a targeted zone within a gastrointestinal tract of the subject, so as to alleviate or dissipate the sensation of straining while defecating.

2. The method of claim 1, said controlling including pre-setting an activation time delay of said capsule, prior to said ingesting.

3. The method of claim 2, wherein the subject is a particular subject and wherein said pre-setting of said activation time delay is according to a measured or estimated transit time of chyme along said gastrointestinal tract of said particular subject.

4. The method of claim 1, wherein the subject is a subject who has experienced at most one of the following symptoms over the preceding 3 months:
fewer than three bowel movements per week;
straining;
lumpy or hard stools;
sensation of anorectal obstruction;
sensation of incomplete defecation; and
manual maneuvering required to defecate.

5. The method of claim 1, wherein the subject is a subject who has experienced at most one of the following symptoms over the preceding 3 months:
fewer than three bowel movements per week;
straining during more than 25% of defecations;
lumpy or hard stools in more than 25% of defecations;
sensation of incomplete defecation in more than 25% of defecations;
sensation of anorectal obstruction in more than 25% of defecations; and
manual maneuvering required to facilitate more 25% of defecations.

6. The method of claim 1, wherein the subject is a constipation free subject.

7. The method of claim 6, wherein said subject suffers from at least one of emotional stress and psychosomatically induced symptoms.

8. The method of claim 1, wherein said ingesting and controlling together form a treatment session, and wherein said method includes administering to the subject at least one said treatment session.

9. The method of claim 8, wherein said administering to the subject at least one treatment session includes administering to the subject a plurality of treatment sessions.

10. The method of claim 9, wherein said administering a plurality of treatment sessions includes administering 1 to 7 said treatment sessions per week, over a treatment period of at least two weeks.

11. A method of increasing a Bristol stool score of fecal matter defecated by a human subject using a gastrointestinal capsule adapted to transit an alimentary canal of the subject, said capsule having:
a housing;
a battery, disposed within said housing; and
a vibrating agitation mechanism, powered by said battery, said vibrating agitation mechanism adapted such that, in a first vibrating mode of operation, said housing exerts vibrations on an environment surrounding said capsule, the method comprising:
(a) ingesting said gastrointestinal capsule; and
(b) controlling said vibrating agitation mechanism such that said first vibrating mode of operation occurs when said capsule is disposed within a targeted zone within a gastrointestinal tract of the subject, so as to increase the Bristol stool score of the fecal matter defecated by the subject.

12. The method of claim 11, said controlling including pre-setting an activation time delay of said capsule, prior to said ingesting.

13. The method of claim 12, wherein the subject is a particular subject and wherein said pre-setting of said activation time delay is according to a measured or estimated transit time of chyme along said gastrointestinal tract of said particular subject.

14. The method of claim 11, wherein the subject is a subject who has experienced at most one of the following symptoms over the preceding 3 months:
fewer than three bowel movements per week;
straining;
lumpy or hard stools;
sensation of anorectal obstruction;
sensation of incomplete defecation; and
manual maneuvering required to defecate.

15. The method of claim 11, wherein the subject is a subject who has experienced at most one of the following symptoms over the preceding 3 months:
fewer than three bowel movements per week;
straining during more than 25% of defecations;
lumpy or hard stools in more than 25% of defecations;
sensation of incomplete defecation in more than 25% of defecations;
sensation of anorectal obstruction in more than 25% of defecations; and
manual maneuvering required to facilitate more 25% of defecations.

16. The method of claim 11, wherein the subject is a constipation free subject.

17. The method of claim 16, wherein said subject suffers from at least one of emotional stress and psychosomatically induced symptoms.

18. The method of claim 11, wherein said ingesting and controlling together form a treatment session, and wherein said method includes administering to the subject at least one said treatment session.

19. The method of claim 18, wherein said administering to the subject at least one treatment session includes administering to the subject a plurality of treatment sessions.

20. The method of claim 19, wherein said administering a plurality of treatment sessions includes administering 1 to 7 said treatment sessions per week, over a treatment period of at least two weeks.
